(19) United States
(12) Patent Application Publication
(10) Pub. No.: US 2026/0330638 A1
(43) Pub. Date: Oct. 22, 2026

(54) ACCELERATION OF MRI EXAMINATIONS OF THE LIVER

(71) Applicants: BAYER AKTIENGESELLSCHAFT, Leverkusen (DE); UNIVERSITÄTSKLINIKUM ESSEN, Essen (DE)

(72) Inventors: Hubertus PIETSCH, Leverkusen (DE); Gregor JOST, Leverkusen (DE); Gesine KNOBLOCH, Leverkusen (DE); Gunnar SCHÜTZ, Leverkusen (DE); Felix Karl KREIS, Leverkusen (DE); Christian LIENERTH, Leverkusen (DE); Johannes HAUBOLD, Essen (DE); René HOSCH, Essen (DE); Felix NENSA, Essen (DE); Michael FORSTING, Essen (DE)

(21) Appl. No.: 19/108,861

(22) PCT Filed: Aug. 29, 2023
(86) PCT No.: PCT/EP2023/073597

§ 371 Date: Mar. 5, 2025

(30) Foreign Application Priority Data
Sep. 7, 2022 (EP) 22194395.4

(51) Int. Cl. G06T 7/00; A61B 5/00; G16H 10/60; G16H 50/20

(52) U.S. Cl. CPC G06T 7/0016; A61B 5/4244; G16H 10/60; G16H 50/20; G06T 2207/10088; G06T 2207/20081; G06T 2207/30056; G06T 2207/30096

(57) ABSTRACT

The present invention is concerned with the acceleration of an MRI examination of the liver by means of a machine learning model. The machine learning model is configured and trained to predict and MRI image of a liver in the hepatobiliary phase after the application of a hepatobiliary contrast agent on the basis of one or more MRI images which were produced at a time in an earlier phase. The present invention relates to a method for training the machine learning model, to a computer-implemented method for predicting the MRI image in the hepatobiliary phase by means of the trained model, and to a computer system and computer program product for carrying out the prediction method.

The present invention relates to the acceleration of an MRI examination of the liver with the aid of a machine learning model. The machine learning model is configured and trained to predict an MRI image of a liver in the hepatobiliary phase after the administration of a hepatobiliary contrast agent on the basis of one or more MRI images which have been generated at a point in time in an earlier phase. The subjects of the present invention are a method for training the machine learning model, a computer-implemented method for predicting the MRI image in the hepatobiliary phase by means of the trained model, and a computer system and a computer program product for carrying out the prediction method.

Tracking processes over time within the body of a human or animal using imaging methods plays an important role, inter alia, in the diagnosis and/or therapy of diseases.

An example that may be mentioned is the detection and differential diagnosis of focal liver lesions by means of dynamic contrast-enhanced magnetic resonance imaging (MRI) using a hepatobiliary contrast agent.

A hepatobiliary contrast agent such as Primovist® can be used for the detection of tumors in the liver. Blood supply to the healthy liver tissue is primarily achieved via the portal vein (vena portae), whereas the liver artery (arteria hepatica) supplies most primary tumors. After intravenous injection of a bolus of contrast agent, it is accordingly possible to observe a time delay between the signal increase in the healthy liver parenchyma and in the tumor.

Besides malignant tumors, what are commonly found in the liver are benign lesions such as cysts, hemangiomas and focal nodular hyperplasias (FNH). Proper planning of therapy requires that these be differentiated from the malignant tumors. Primovist® can be used for the detection and differentiation of benign and malignant focal liver lesions. By means of T1-weighted MRI, it provides information about the character of these lesions. Differentiation is achieved by utilizing the difference in blood supply to liver and tumor and the time profile of contrast enhancement.

In the case of the contrast enhancement achieved by Primovist® during the wash-in phase, typical perfusion patterns are observed, which provide information for the characterization of the lesions. Depicting the vascularization helps to characterize the lesion types and to determine the spatial relationship between tumor and blood vessels.

In the case of T1-weighted MRI images, Primovist® results, 10-20 minutes after the injection (in the hepatobiliary phase), in a distinct signal enhancement in the healthy liver parenchyma, whereas lesions containing no hepatocytes or only a few hepatocytes, for example metastases or moderately to poorly differentiated hepatocellular carcinomas (HCCs), appear as regions that are more T1 hypo-intense, whereas for example focal nodular hyperplasias accumulate contrast agent in this phase.

Tracking the spread of the contrast agent over time thus provides a good way of detecting and differentially diagnosing focal liver lesions; however, the examination extends over a comparatively long period of time. Over this period of time, movements by the patient should be largely avoided in order to minimize movement artifacts in the MRI images. The lengthy restriction of movement can be unpleasant for a patient.

Laid-open application WO2021/052896A1 proposes that one or more MRI images during the hepatobiliary phase be generated not by measurement, but calculated (predicted) on the basis of MRI images from a plurality of preceding phases in order to shorten the time spent by the patient in the MRI scanner.

It has surprisingly been found that MRI images from a plurality of different phases are not required to predict an MRI image of the liver in the hepatobiliary phase. It has surprisingly been found that the quality of the predicted MRI image is equivalent if the prediction is carried out solely on the basis of one or more contrast-agent-amplified MRI images, which represent(s) the liver at a point in time in the transitional phase, and optionally one or more native MRI images.

A first subject matter of the present invention is therefore a method for training a machine learning model, wherein the method comprises:
receiving and/or providing training data, wherein the training data comprise input data and target data for each examination object of a plurality of examination objects,
wherein the input data comprise MRI images, wherein the MRI images are restricted to:
   at least one first MRI image, wherein the at least one first MRI image represents a liver or a part of the liver of the examination object at a point in time in the transitional phase after an administration of a hepatobiliary contrast agent, and
   optionally at least one native MRI image of the liver or the part of the liver of the examination object without contrast agent,
wherein the target data comprise a second MRI image, wherein the second MRI image represents the liver or the part of the liver of the examination object at a point in time in the hepatobiliary phase after the administration of the hepatobiliary contrast agent,
training the machine learning model, wherein the machine learning model is configured to generate a predicted second MRI image on the basis of at least one first MRI image, optionally at least one native MRI image, and model parameters, wherein the training comprises, for each examination object of the plurality of examination objects:
   inputting the input data into the machine learning model,
   receiving a predicted second MRI image from the machine learning model,
   calculating a deviation between the second MRI image and the predicted second MRI image,
   reducing the deviation by modifying the model parameters,
storing and/or outputting the trained machine learning model and/or transmitting the trained machine learning model to a separate computer system and/or using the trained machine learning model for prediction.

A further subject matter of the present invention is a computer-implemented method for predicting an MRI image, wherein the method comprises:
receiving patient data, wherein the patient data comprise at least one MRI image, wherein the at least one MRI image is restricted to at least one first MRI image and optionally at least one native MRI image, wherein the at least one first MRI image represents a liver or a part of the liver of a patient at a point in time in the transitional phase after an administration of a hepatobiliary contrast agent, wherein the optional at least one native MRI image represents the liver or the part of the liver of the patient without contrast agent,
inputting the patient data into a trained machine learning model, wherein the machine learning model was trained on the basis of training data to predict a second MRI image on the basis of at least one first MRI image and optionally at least one native MRI image, wherein the training data comprise input data and target data for each examination object of a plurality of examination objects,
wherein the input data comprise MRI images, wherein the MRI images are restricted to:
   at least one first MRI image, wherein the at least one first MRI image represents a liver or a part of the liver of the examination object at a point in time in the transitional phase after an administration of a hepatobiliary contrast agent, and
   optionally at least one native MRI image of the liver or the part of the liver of the examination object without contrast agent,
wherein the target data comprise a second MRI image, wherein the second MRI image represents the liver or the part of the liver of the examination object at a point in time in the hepatobiliary phase after the administration of the hepatobiliary contrast agent,
receiving a predicted MRI image from the trained machine learning model, wherein the predicted MRI image represents the liver or the part of the liver of the patient at a point in time in the hepatobiliary phase after the administration of the hepatobiliary contrast agent,
outputting and/or storing the predicted MRI image and/or transmitting the predicted MRI image to a separate computer system.

A further subject matter of the present invention is a computer system, comprising
an input unit,
a control and calculation unit, and
an output unit,
   wherein the control and calculation unit is configured
to cause the input unit to receive patient data, wherein the patient data comprise at least one MRI image, wherein the at least one MRI image is restricted to at least one first MRI image and optionally at least one native MRI image, wherein the at least one first MRI image represents a liver or a part of the liver of a patient at a point in time in the transitional phase after an administration of a hepatobiliary contrast agent, wherein the optional at least one native MRI image represents the liver or the part of the liver of the patient without contrast agent,
to input the patient data into a trained machine learning model, wherein the machine learning model was trained on the basis of training data to predict a second MRI image on the basis of at least one first MRI image and optionally at least one native MRI image, wherein the training data comprise input data and target data for each examination object of a plurality of examination objects,
wherein the input data comprise MRI images, wherein the MRI images are restricted to:
   at least one first MRI image, wherein the at least one first MRI image represents a liver or a part of the liver of the examination object at a point in time in the transitional phase after an administration of a hepatobiliary contrast agent, and
   optionally at least one native MRI image of the liver or the part of the liver of the examination object without contrast agent,
wherein the target data comprise a second MRI image, wherein the second MRI image represents the liver or the part of the liver of the examination object at a point in time in the hepatobiliary phase after the administration of the hepatobiliary contrast agent,
to receive a predicted MRI image from the trained machine learning model, wherein the predicted MRI image represents the liver or the part of the liver of the patient at a point in time in the hepatobiliary phase after the administration of the hepatobiliary contrast agent,
to cause the output unit to output the predicted MRI image and/or to store it and/or to transmit it to a separate computer system.

A further subject matter of the present invention is a computer program product comprising a data memory in which a computer program that can be loaded into a working memory of a computer system is stored, where it causes the computer system to execute the following steps:
receiving patient data, wherein the patient data comprise at least one MRI image, wherein the at least one MRI image is restricted to at least one first MRI image and optionally at least one native MRI image, wherein the at least one first MRI image represents a liver or a part of the liver of a patient at a point in time in the transitional phase after an administration of a hepatobiliary contrast agent, wherein the optional at least one native MRI image represents the liver or the part of the liver of the patient without contrast agent,
inputting the patient data into a trained machine learning model, wherein the machine learning model was trained on the basis of training data to predict a second MRI image on the basis of at least one first MRI image and optionally at least one native MRI image, wherein the training data comprise input data and target data for each examination object of a plurality of examination objects,
wherein the input data comprise MRI images, wherein the MRI images are restricted to:
   at least one first MRI image, wherein the at least one first MRI image represents a liver or a part of the liver of the examination object at a point in time in the transitional phase after an administration of a hepatobiliary contrast agent, and
   optionally at least one native MRI image of the liver or the part of the liver of the examination object without contrast agent,
wherein the target data comprise a second MRI image, wherein the second MRI image represents the liver or the part of the liver of the examination object at a point in time in the hepatobiliary phase after the administration of the hepatobiliary contrast agent,
receiving a predicted MRI image from the trained machine learning model, wherein the predicted MRI image represents the liver or the part of the liver of the patient at a point in time in the hepatobiliary phase after the administration of the hepatobiliary contrast agent,
outputting and/or storing the predicted MRI image and/or transmitting the predicted MRI image to a separate computer system.

A further subject matter of the present invention is a hepatobiliary contrast agent for use in an MRI examination method, comprising:
receiving and/or generating patient data, wherein the patient data comprise at least one MRI image, wherein the at least one MRI image is restricted to at least one first MRI image and optionally at least one native MRI image, wherein the at least one first MRI image represents a liver or a part of the liver of a patient at a point in time in the transitional phase after an administration of the hepatobiliary contrast agent, wherein the optional at least one native MRI image represents the liver or the part of the liver of the patient without contrast agent,
inputting the patient data into a trained machine learning model, wherein the machine learning model was trained on the basis of training data to predict a second MRI image on the basis of at least one first MRI image and optionally at least one native MRI image, wherein the training data comprise input data and target data for each examination object of a plurality of examination objects,
wherein the input data comprise MRI images, wherein the MRI images are restricted to:
   at least one first MRI image, wherein the at least one first MRI image represents a liver or a part of the liver of the examination object at a point in time in the transitional phase after an administration of a hepatobiliary contrast agent, and
   optionally at least one native MRI image of the liver or the part of the liver of the examination object without contrast agent,
wherein the target data comprise a second MRI image, wherein the second MRI image represents the liver or the part of the liver of the examination object at a point in time in the hepatobiliary phase after the administration of the hepatobiliary contrast agent,
receiving a predicted MRI image from the trained machine learning model, wherein the predicted MRI image represents the liver or the part of the liver of the patient at a point in time in the hepatobiliary phase after the administration of the hepatobiliary contrast agent,
outputting and/or storing the predicted MRI image and/or transmitting the predicted MRI image to a separate computer system.

A further subject matter of the present invention is a use of a hepatobiliary contrast agent in an MRI examination method, comprising:
receiving patient data, wherein the patient data comprise at least one MRI image, wherein the at least one MRI image is restricted to at least one first MRI image and optionally at least one native MRI image, wherein the at least one first MRI image represents a liver or a part of the liver of a patient at a point in time in the transitional phase after an administration of the hepatobiliary contrast agent, wherein the optional at least one native MRI image represents the liver or the part of the liver of the patient without contrast agent,
inputting the patient data into a trained machine learning model, wherein the machine learning model was trained on the basis of training data to predict a second MRI image on the basis of at least one first MRI image and optionally at least one native MRI image, wherein the training data comprise input data and target data for each examination object of a plurality of examination objects,
wherein the input data comprise MRI images, wherein the MRI images are restricted to:
   at least one first MRI image, wherein the at least one first MRI image represents a liver or a part of the liver of the examination object at a point in time in the transitional phase after an administration of a hepatobiliary contrast agent, and
   optionally at least one native MRI image of the liver or the part of the liver of the examination object without contrast agent,
wherein the target data comprise a second MRI image, wherein the second MRI image represents the liver or the part of the liver of the examination object at a point in time in the hepatobiliary phase after the administration of the hepatobiliary contrast agent,
receiving a predicted MRI image from the trained machine learning model, wherein the predicted MRI image represents the liver or the part of the liver of the patient at a point in time in the hepatobiliary phase after the administration of the hepatobiliary contrast agent,
outputting and/or storing the predicted MRI image and/or transmitting the predicted MRI image to a separate computer system.

A further subject matter of the present invention is a kit comprising a hepatobiliary contrast agent and a computer program product, wherein the computer program product comprises a data memory in which a computer program that can be loaded into a working memory of a computer system is stored, where it causes the computer system to execute the following steps:
receiving patient data, wherein the patient data comprise at least one MRI image, wherein the at least one MRI image is restricted to at least one first MRI image and optionally at least one native MRI image, wherein the at least one first MRI image represents a liver or a part of the liver of a patient at a point in time in the transitional phase after an administration of the hepatobiliary contrast agent, wherein the optional at least one native MRI image represents the liver or the part of the liver of the patient without contrast agent,
inputting the patient data into a trained machine learning model, wherein the machine learning model was trained on the basis of training data to predict a second MRI image on the basis of at least one first MRI image and optionally at least one native MRI image, wherein the training data comprise input data and target data for each examination object of a plurality of examination objects,
wherein the input data comprise MRI images, wherein the MRI images are restricted to:
   at least one first MRI image, wherein the at least one first MRI image represents a liver or a part of the liver of the examination object at a point in time in the transitional phase after an administration of a hepatobiliary contrast agent, and
   optionally at least one native MRI image of the liver or the part of the liver of the examination object without contrast agent,
wherein the target data comprise a second MRI image, wherein the second MRI image represents the liver or the part of the liver of the examination object at a point in time in the hepatobiliary phase after the administration of the hepatobiliary contrast agent,
receiving a predicted MRI image from the trained machine learning model, wherein the predicted MRI image represents the liver or the part of the liver of the patient at a point in time in the hepatobiliary phase after the administration of the hepatobiliary contrast agent,
outputting and/or storing the predicted MRI image and/or transmitting the predicted MRI image to a separate computer system.

The invention will be more particularly elucidated below without distinguishing between the subjects of the invention (training method, prediction method, computer system, computer program product, use, contrast agent for use, kit). Rather, the elucidations that follow are intended to apply analogously to all subjects of the invention, irrespective of the context (training method, prediction method, computer system, computer program product, use, contrast agent for use, kit) in which they occur.

Where steps are stated in an order in the present description or in the claims, this does not necessarily mean that the invention is limited to the order stated. Instead, it is conceivable that the steps are also executed in a different order or else in parallel with one another, the exception being when one step builds on another step, thereby making it imperative that the step building on the previous step be executed next (which will however become clear in the individual case). The orders stated are thus preferred embodiments of the invention.

In certain places the invention will be more particularly elucidated with reference to drawings. The drawings show specific embodiments having specific features and combinations of features, which are intended primarily for illustrative purposes; the invention is not to be understood as being limited to the features and combinations of features shown in the drawings. Furthermore, statements made in the description of the drawings in relation to features and combinations of features are intended to be generally applicable, that is to say applicable to other embodiments too and not limited to the embodiments shown.

With the aid of the present invention, it is possible to predict an MRI image of an examination region of an examination object. In this description, the prediction of an MRI image is synonymously also referred to as the generation of a predicted MRI image.

The “examination object” is normally a living being, preferably a mammal, very particularly preferably a human. The examination object is also referred to as a patient in this description.

The “examination region” is a liver or a part of the liver of the examination object.

In a preferred embodiment of the present invention, the examination region is the liver or part of the liver of a human.

The examination region, also referred to as the field of view (FOV), is in particular a volume that is imaged in radiological images. The examination region is typically defined by a radiologist, for example on a localizer image. It is of course also possible for the examination region to be alternatively or additionally defined in an automated manner, for example on the basis of a selected protocol.

Magnetic resonance imaging, MRI for short, is an imaging method which is used especially in medical diagnostics for depicting structure and function of the tissues and organs in the human or animal body.

In MRI, the magnetic moments of protons in an examination object are aligned in a basic magnetic field, with the result that there is a macroscopic magnetization along a longitudinal direction. This is then deflected from the resting position by irradiation with high-frequency (HF) pulses (excitation). The return of the excited states to the resting position (relaxation), or magnetization dynamics, is then detected as relaxation signals by means of one or more HF receiver coils.

For spatial encoding, rapidly switched magnetic gradient fields are superimposed on the basic magnetic field. The captured relaxation signals, or the detected MRI data, are initially present as raw data in frequency space, and can be transformed by subsequent inverse Fourier transform into real space (image space).

The predicted MRI image can be a representation of the examination region in real space (image space) or a representation in frequency space or another representation. MRI images used for training the machine learning model and for prediction are typically present in a real space representation or in a form which can be converted (transformed) into a real space representation.

An MRI image in the meaning of the present invention can be a two-dimensional, three-dimensional, or higher-dimensional representation. Usually, two-dimensional (2D) tomograms (slice images) are present, or a stack of two-dimensional tomograms (slice images) are present, or a three-dimensional (3D) representation is present.

The MRI images are usually in digital form. The term “digital” means that the representations can be processed by a machine, generally a computer system. “Processing” is understood to mean the known methods for electronic data processing (EDP). An example of a typical format for a digital MRI image is the DICOM format (DICOM: Digital Imaging and Communications in Medicine)—an open standard for storing and exchanging information in medical image-data management.

In the interests of simpler illustration, the invention will be elucidated at some points in the present description on the basis of the presence of two-dimensional images, without any wish, however, to restrict the invention to two-dimensional images. It is clear to a person skilled in the art how it is possible to apply what is respectively described to stacks of two-dimensional images, to 3D images, or to representations in frequency space.

The predicted MRI image represents the liver or the part of the liver of the examination object in the hepatobiliary phase after the administration of a hepatobiliary contrast agent.

A hepatobiliary contrast agent is understood to mean a contrast agent which is specifically absorbed by healthy liver cells, the hepatocytes.

Examples of hepatobiliary contrast agents are contrast agents based on gadoxetic acid. They are described for example in U.S. Pat. No. 6,039,931A. They are commercially available under the trade names Primovist® or Eovist®, for example.

The contrast-enhancing effect of Primovist®/Eovist® is mediated by the stable gadolinium complex Gd-EOB-DTPA (gadolinium ethoxybenzyl diethylenetriaminepentaacetic acid). DTPA forms with the paramagnetic gadolinium ion a complex that has extremely high thermodynamic stability. The ethoxybenzyl (EOB) radical is the mediator of the hepatobiliary uptake of the contrast agent.

A further MRI contrast agent having a lower uptake into the hepatocytes is gadobenate dimeglumine (Multihance®).

Further hepatobiliary contrast agents are described, inter alia, in WO2022/194777.

In one embodiment of the present disclosure, the contrast agent used is a substance or a substance mixture having gadoxetic acid or a salt of gadoxetic acid as contrast-enhancing active substance. Very particularly preferably it is the disodium salt of gadoxetic acid (Gd-EOB-DTPA disodium).

In one embodiment of the present disclosure, the contrast agent is an agent that includes gadolinium 2,2′,2″-(10-{1-carboxy-2-[2-(4-ethoxyphenyl)ethoxy]ethyl}-1,4,7,10-tetraazacyclododecane-1,4,7-triyl)triacetate (see for example WO 2022/194777, example 1).

In one embodiment of the present disclosure, the contrast agent is an agent that includes gadolinium 2,2′,2″-{10-[1-carboxy-2-{4-[2-(2-ethoxyethoxy)ethoxy]phenyl}ethyl]- 1,4,7,10-tetraazacyclododecane-1,4,7-triyl}triacetate (see for example WO 2022/194777, example 2).

In one embodiment of the present disclosure, the contrast agent is an agent that includes gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate (see for example WO 2022/194777, example 4).

In one embodiment of the present disclosure, the contrast agent is an agent that includes gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate) (see for example WO2022/194777, example 15).

In one embodiment of the present disclosure, the contrast agent is an agent that includes gadolinium 2,2′,2″-{10-[(1S)-4-(4-butoxyphenyl)-1-carboxybutyl]-1,4,7,10-tetraazacyclododecane-1,4,7-triyl}triacetate (see for example WO 2022/194777, example 31).

After the intravenous administration of the hepatobiliary contrast agent in the form of a bolus into an arm vein, the contrast agent reaches the liver first via the arteries. These are depicted with signal enhancement in T1-weighted MRI images. The phase in which the liver arteries are depicted with signal enhancement in T1-weighted MRI images is referred to as the “arterial phase”.

Subsequently, the contrast agent reaches the liver via the liver veins. While the signal enhancement in the liver arteries is already decreasing, the signal enhancement in the liver veins is reaching a maximum. The phase in which the liver veins are depicted with signal enhancement in T1-weighted MRI images is referred to as “portal-venous phase”. This phase can already begin during the arterial phase and overlap therewith.

The portal-venous phase is followed by the “transitional phase”, in which the signal enhancement in the liver arteries drops further, and the signal enhancement in the liver veins likewise drops. When using a hepatobiliary contrast agent, the signal enhancement in the healthy liver cells gradually rises in the transitional phase.

The arterial phase, the portal-venous phase, and the transitional phase are also referred to collectively as the “dynamic phase”.

10-20 minutes after being injected, a hepatobiliary contrast agent results in a distinct signal enhancement in the healthy liver parenchyma. This phase is referred to as the “hepatobiliary phase”. The contrast agent is eliminated only slowly from the liver cells; accordingly, the hepatobiliary phase can last for two hours and longer.

The stated phases are for example described in more detail in the following publications: J. Magn. Reson. Imaging, 2012, 35(3): 492-511, doi: 10.1002/jmri.22833; Clujul Medical, 2015, Vol. 88 no. 4: 438-448, DOI: 10.15386/cjmed-414; Journal of Hepatology, 2019, Vol. 71:534-542, http://dx.doi.org/10.1016/j.jhep.2019.05.005).

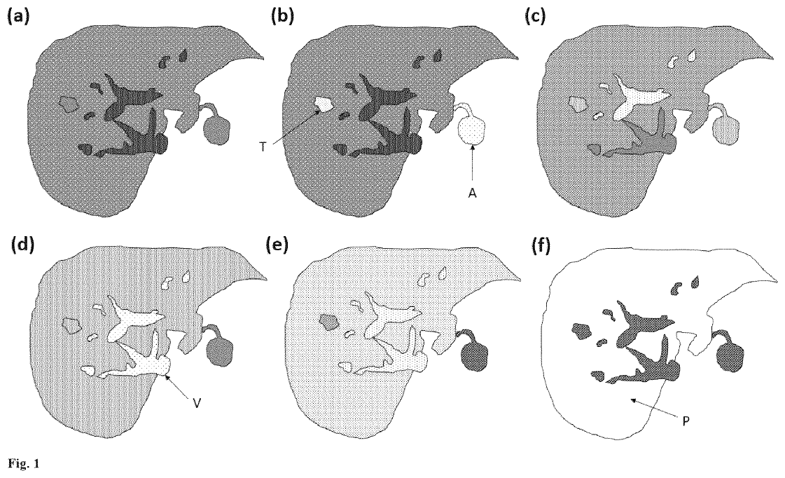

FIG. 1 shows schematically and by way of example a plurality of T1-weighted MRI images of a part of a liver of an examination object during the dynamic phase and the hepatobiliary phase. Depicted in each of FIGS. 1(a), 1(b), 1(c), 1(d), 1(e) and 1(f) is the same cross section through the liver, at different points in time. The reference signs included in FIGS. 1(b), 1(d), and 1(f) apply to all of FIGS. 1(a), 1(b), 1(c), 1(d), 1(e), and 1(f); they are each included only once merely for the sake of clarity.

FIG. 1(a) shows the cross section through the liver before the intravenous administration of a hepatobiliary contrast agent (native MRI image). At a point in time between the points in time depicted by FIGS. 1(a) and 1(b), a hepatobiliary contrast agent was administered intravenously as a bolus. This reaches the liver via the liver artery (A) in FIG. 1(b). Accordingly, the liver artery is depicted with signal enhancement (arterial phase). A tumor (T), which is supplied with blood mainly via arteries, likewise stands out from the liver-cell tissue as a lighter (signal-enhanced) region. At the point in time depicted in FIG. 1(c), the contrast agent reaches the liver via the veins. In FIG. 1(d), the venous blood vessels (V) stand out from the liver tissue as light (signal-enhanced) regions (venous phase). At the same time, the signal intensity in the healthy liver cells, which are supplied with contrast agent mainly via the veins, continuously rises (FIG. 1(c)→1(d)→1(e)∛1(f)). FIG. 1(e) shows a T1-weighted MRI image in the transitional phase. A signal enhancement can already be seen in the healthy liver cells (P), while the signal already drops again in the veins. In the hepatobiliary phase depicted in FIG. 4(f), the liver cells (P) are depicted with signal enhancement; the blood vessels and the tumor no longer have contrast agent and are accordingly depicted darkly.

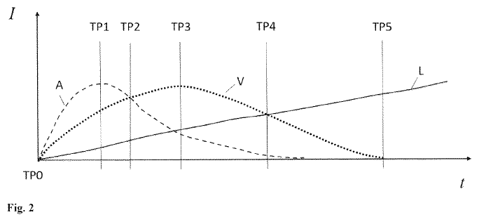

FIG. 2 schematically shows the temporal profile (t=time) of the signal intensities I that are caused by a hepatobiliary contrast agent in liver arteries (A), liver veins (V), and healthy liver cells (P) in a contrast-enhanced MRI examination. The signal intensity I has a positive correlation with the concentration of the contrast agent in the stated regions. Upon an intravenous bolus injection, the concentration of the contrast agent rises in the liver arteries (A) first of all (dashed curve). The concentration passes through a maximum and then drops. The concentration in the liver veins (V) rises more slowly than in the liver arteries and reaches its maximum later (dotted curve). The concentration of the contrast agent in the healthy liver cells (P) rises slowly (solid curve) and reaches its maximum only at a very much later point in time (not depicted in FIG. 1). A few characteristic points in time can be defined: At point in time TP0, contrast agent is administered intravenously as a bolus. Since the administration of a contrast agent itself requires a certain time span, point in time TP0 preferably defines the point in time at which the administration is completed, i.e. at which contrast agent is completely introduced into the examination object. At point in time TP1, the signal intensity of the contrast agent in the liver arteries (A) reaches its maximum. At point in time TP2, the curves of the signal intensities for the liver arteries (A) and the liver veins (V) intersect. At point in time TP3, the signal intensity of the contrast agent in the liver veins (V) passes through its maximum. At point in time TP4, the curves of the signal intensities for the liver arteries (A) and the healthy liver cells (P) intersect. At point in time TP5, the concentrations in the liver arteries (A) and the liver veins (V) have dropped to a level at which they no longer cause a measurable contrast enhancement.

The prediction of an MRI image in the hepatobiliary phase is carried out on the basis of at least one MRI image, wherein the at least one MRI image is restricted to i) at least one first MRI image in the transitional phase and ii) optionally at least one native MRI image. In other words: no MRI images from the arterial phase and/or the portal-venous phase are used for the prediction. The at least one native MRI image shows the liver or the part of the liver without contrast agent. It is preferably generated before the administration of the hepatobiliary contrast agent.

As described in international patent application WO2022223383A1 (PCT/EP2022/059836), image registration errors can be reduced if not only one native MRI image is used for the prediction, but rather more than one native MRI image is used (e.g. two or three native MRI images). If more than one native MRI image is used, the machine learning model learns during the training to compensate for movement artifacts.

The prediction of the MRI image in the hepatobiliary phase takes place with the aid of a machine learning model.

A “machine learning model” can be understood as a computer-implemented data processing architecture. The model is able to receive input data and to supply output data on the basis of said input data and model parameters. The model can learn a relationship between the input data and the output data by means of training. During training, the model parameters can be adjusted so as to supply a desired output for a particular input.

During the training of such a model, the model is presented with training data from which it can learn. The trained machine learning model is the result of the training process. Besides input data, the training data include the correct output data (target data) that are to be generated by the model on the basis of the input data. During training, patterns that map the input data onto the target data are recognized.

In the training process, the input data of the training data are input into the model, and the model generates output data. The output data are compared with the target data. Model parameters are altered so as to reduce the differences between the output data and the target data to a (defined) minimum.

During training, a loss function can be used to control the training process and to assess the prediction quality of the model. The loss function can be chosen such that it “rewards” a desired relationship between output data and target data and/or “punishes” an undesired relationship between output data and target data. Such a relationship can be e.g. a similarity, a dissimilarity, or some other relationship.

A loss function can be used to calculate losses for a given pair made up of output data and target data. The goal of the training process can consist in altering (adjusting) the parameters of the machine learning model so as to reduce the loss value for all pairs of the training data set to a (defined) minimum.

A loss function can quantify e.g. the difference between the output data of the model for specific input data and the target data. For example, if the output data and the target data are numbers, the loss function can be the absolute difference between these numbers. In this case, a high absolute value of the loss function can mean that one or more model parameters have to be changed to a great extent.

In the case of output data in the form of vectors, for example difference metrics between vectors such as the mean squared error, a cosine distance, a norm of the difference vector such as a Euclidean distance, a Chebyshev distance, an Lp norm of a difference vector, a weighted norm, or any other type of difference metric of two vectors can be chosen as the loss function.

In the case of higher-dimensional outputs, such as two-dimensional, three-dimensional or higher-dimensional outputs, an element-by-element difference metric can for example be used. Alternatively or additionally, the output data may be transformed before calculation of a loss value, e.g. into a one-dimensional vector.

In the present case, the training data comprise MRI images for each examination object of a plurality of examination objects, wherein the MRI images are restricted to:
at least one first MRI image, wherein the at least one first MRI image represents a liver or a part of the liver of the examination object at a point in time in the transitional phase after an administration of a hepatobiliary contrast agent, and
optionally at least one native MRI image of the liver or the part of the liver of the examination object without contrast agent and
a second MRI image, wherein the second MRI image represents the liver or the part of the liver of the examination object in the hepatobiliary phase after the administration of the hepatobiliary contrast agent.

The term “plurality of examination objects” means at least ten, preferably at least one hundred, examination objects.

The at least one first MRI image and the optional at least one native MRI image are used as input data for the machine learning model. The second MRI image is used as target data.

Preferably, the at least one first MRI image is at least one representation of the examination region of the examination object at a point in time in the range of 3 minutes to 6 minutes after the administration of the hepatobiliary contrast agent. Particularly preferably, the at least one first MRI image is at least one representation of the examination region of the examination object at a point in time in the range of minutes to 5 minutes after the administration of the hepatobiliary contrast agent. Particularly preferably, the at least one first MRI image is at least one representation of the examination region of the examination object at a point in time in the range between the points in time TP3 and TP5 shown in FIG. 2.

“Administration” is understood in this description preferably as the intravenous administration of the contrast agent as a bolus into an arm vein of the patient.

Gadoxetate disodium (GD, Primovist®) is approved for example at a dose of 0.1 ml/kg body weight (BW) (0.025 mmol/kg BW Gd). The recommended administration of GD comprises an undiluted intravenous bolus injection at a flow rate of approximately 2 ml/second, followed by flushing of the i.v. cannula with a physiological saline solution.

The at least one first MRI image preferably represents the examination region at a defined point in time or at a defined time span, wherein the time span corresponds to the duration of the recording of raw data to generate the at least one first MRI image.

The at least one first MRI image is preferably an MRI image which was generated at a point in time after point in time TP3 (see FIG. 2).

Preferably, the at least one first MRI image is an MRI image which was generated at a point in time after point in time TP3 and before point in time TP5 (see FIG. 2).

Preferably, the at least one first MRI image is an MRI image which was generated at a point in time between 30 seconds before point in time TP4 and 30 seconds after point in time TP4 (see FIG. 2).

Preferably, the at least one first MRI image comprises a T1-weighted MRI image of the examination region.

Preferably, the at least one first MRI image is the result of a Dixon sequence, preferably a T1-weighted Dixon sequence, preferably with in-and opposed-phase (water to fat) recording technology (see e.g. A. S. Shetty: In-Phase and Opposed-Phase Imaging: Applications of Chemical Shift and Magnetic Susceptibility in the Chest and Abdomen, RadioGraphics 2019, 39:115-135) and/or the at least one first MRI image comprises such a result.

Preferably, the at least one first MRI image comprises an in-phase image and an opposed-phase image and also a fat-only image and a water-only image.

Preferably, the at least one native MRI image comprises a T1-weighted MRI image of the examination region.

Preferably, the at least one native MRI image is the result of a Dixon sequence, preferably a T1-weighted Dixon sequence, preferably with in-and opposed-phase (water to fat) recording technology.

Preferably, the at least one native MRI image is a water-only image of the examination region or comprises such an image.

Preferably, the second MRI image is a representation of the liver at a point in time in the range from 10 minutes to 30 minutes after the administration of the hepatobiliary contrast agent. Particularly preferably, the second MRI image is a representation of the examination region of the examination object at a point in time in the range of 15 minutes to 20 minutes after the administration of the hepatobiliary contrast agent.

The second MRI image preferably represents the examination region at a defined point in time or at a defined time span in the hepatobiliary phase, wherein the time span corresponds to the duration of the recording of raw data to generate the second MRI image.

Preferably, the second MRI image is a T1-weighted MRI image. Preferably, the second MRI image is a water-only image of the examination region or comprises such an image.

Preferably, the same sequences were used for the recording of the at least one first MRI image, the optional at least one native MRI image, and the second MRI image and identical contrast-determining measuring parameters (e.g. repetition time, echo times, flip angle, fat-suppression methods) were used in order that the differences in the image contrast originate only from the contrast agent.

Before MRI images are supplied to the machine learning model, a registration is typically carried out.

Such a registration (also called “co-registration” or image registration) is a process in digital image processing and serves to bring two or more images of the same scene, or at least similar scenes, in harmony with one another in the best possible way. One of the images is defined as the reference image and the others are called object images. In order to optimally match these object images with the reference image, a compensating transformation is calculated. The images to be registered differ from one another because they were recorded from different positions, at different points in time, or using different sensors.

In the case of the MRI images of the present invention, they were recorded at different points in time.

The goal of image registration is thus to find that transformation which brings a given object image into harmony with the reference image in the best possible way. The goal is that, where possible, each pixel/voxel of an image represents the same examination region in an examination object as the pixel/voxel of a different (co-registered) image having the same coordinates.

Methods for image registration are described in the prior art (see e.g.: E. H. Seeley et al.: Co-registration of multi-modality imaging allows for comprehensive analysis of tumor-induced bone disease, Bone 2014, 61, 208-216; C. Bhushan et al.: Co-registration and distortion correction of diffusion and anatomical images based on inverse contrast normalization, Neuroimage 2015, 15, 115:269-80; US20200214619; US20090135191; EP3639272A).

It is possible that the MRI images are subjected to further image processing methods, such as e.g. filtering, upsampling, downsampling, reduction to a region of interest, and/or others.

It is conceivable that the prediction is not exclusively based on the at least one first MRI image and optionally on the at least one native MRI image, but rather that further data are used as further input data. Such further data can comprise information on the examination object (e.g. age, sex, height, weight, body mass index, resting heart rate, heart rate variability, body temperature, information about the lifestyle of the examination object, such as e.g. alcohol consumption, smoking, and/or physical activity and/or the diet of the examination object, medical intervention parameters such as regular medication, occasional medication, or other earlier or current medical interventions and/or other information about earlier and current treatment of the examination object and reported health states and/or combinations thereof), information about the examination region (e.g. size and/or shape of the examination region and/or the location of the examination region within the examination object) and/or information about the at least one MRI image (e.g. under which conditions it has been generated and/or which measurement parameters and/or measurement sequences were used in order to generate it).

During the training of the machine learning model, for each examination object of the plurality of examination objects, the at least one first MRI image and optionally the at least one native MRI image and optionally further data are input as input data into the machine learning model. The machine learning model is configured to generate output data on the basis of the input data and on the basis of model parameters. The output data represent a predicted second MRI image, i.e. a predicted MRI image of the examination region of the examination object in the hepatobiliary phase.

The predicted second MRI image is compared for each examination object with the respective (preferably measured) second MRI image. The deviations can be quantified with the aid of a loss function. Suitable loss functions for quantifying deviations between two MRI images are for example L1 loss function, L2 loss function, Lp loss function, structural similarity index measure (SSIM), VGG loss function, perceptual loss function, or a combination of the aforementioned functions, or other loss functions. Further details on loss functions can be found e.g. in the scientific literature (see e.g.: R. Mechrez et al.: The Contextual Loss for Image Transformation with Non-Aligned Data, 2018, arXiv:1803.02077v 4; H. Zhao et al.: Loss Functions for Image Restoration with Neural Networks, 2018, arXiv:1511.08861v3).

With the aid of a gradient method or some other optimization method, the model parameters can be modified such that the loss is reduced for all pairs and preferably falls below a predefined threshold value and/or is reduced to a defined minimum.

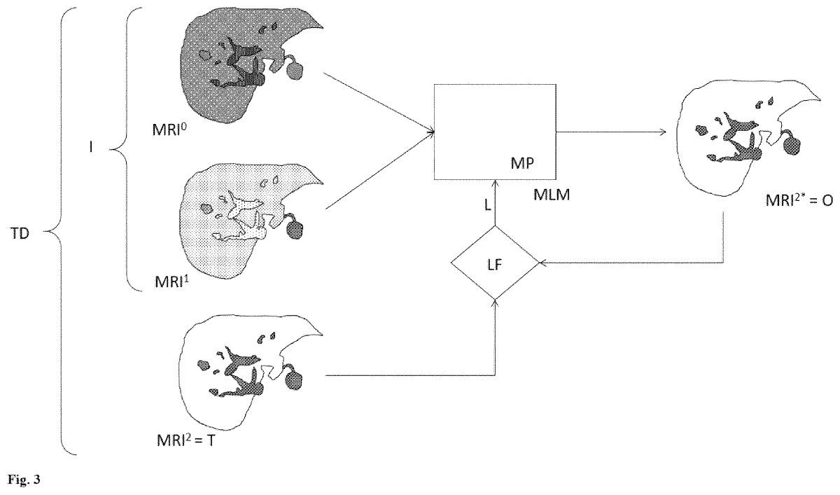

FIG. 3 shows by way of example and schematically an embodiment of the method for training the machine learning model.

The machine learning model (MLM) is trained with the aid of training data (TD). The training data for each examination object of a plurality of examination objects typically comprise input data and target data. Only one data set of an examination object, which consists of the input data (I) and the target data (T), is shown in FIG. 3. The input data (I) consist in the present example of at least one first MRI image (MRI¹), which represents the liver of the examination object at a point in time in the transitional phase after the administration of a hepatobiliary contrast agent, and of at least one native MRI image (MRI⁰), which represents the liver of the examination object without contrast agent. As described, the use of one or more native MRI images is optional. As described, the input data (I) can comprise further data, wherein the further data do not comprise any MRI images of a phase other than the transitional phase and optionally the native phase, however. The target data (T) consists in the present example of a second MRI image (MRI²), which represents the liver at a point in time in the hepatobiliary phase after the administration of the contrast agent.

The at least one first MRI image (MRI¹) can be for example the MRI image shown in FIG. 1(e). The at least one native MRI image (MRI⁰) can be for example the MRI image shown in FIG. 1(a). The second MRI image (MRI²) can be for example the MRI image shown in FIG. 1(f).

The input data (I) are fed to the machine learning model (MLM). The machine learning model (MLM) is configured to generate output data (O) on the basis of the input data (I) and on the basis of model parameters (MP). The output data (O) represent a predicted second MRI image (MRI^{2*}), which represents the liver of the examination object at a point in time in the transitional phase after the administration of a hepatobiliary contrast agent.

By means of a loss function (LF), deviations between the predicted second MRI image (MRI^{2*}) and the second MRI image (MRI²) of the training data are quantified. A loss (L) can be calculated for each pair made up of input data and output data. The loss (L) can be used in an optimization method (e.g. a gradient method), in order to modify model parameters (MP), in order that the loss (L) is reduced to a defined minimum.

If the model has been trained on the basis of a plurality of pairs made up of input data and target data and the loss (L) has reached a defined minimum for all pairs, the trained machine learning model can be used for the prediction. This is shown by way of example and schematically in FIG. 4.

FIG. 4 shows by way of example and schematically an embodiment of the method for predicting an MRI image of a liver or a part of the liver of a patient, wherein the MRI image represents the liver or the part of the liver in a hepatobiliary phase after the administration of a hepatobiliary contrast agent.

The prediction is carried out with the aid of a trained machine learning model (MLM^{t}). The superscript “t” in MLM^{t} is to illustrate that it is the trained model. The trained machine learning model (MLM^{t}) shown in FIG. 4 can have been trained for example as described in relation to FIG. 3.

The prediction is carried out on the basis of patient data (PD). The patient data (PD) comprise at least one first MRI image (MRI_{P}¹), wherein the at least one first MRI image (MRI_{P}¹) represents the liver or the part of the liver of the patient in the transitional phase after the administration of the hepatobiliary contrast agent. In the present example, the patient data (PD) furthermore comprise at least one native MRI image (MRI_{P}⁰). The at least one native MRI image (MRI_{P}⁰) shows the liver or the part of the liver without a contrast agent.

The MRI images which are used or are generated in the case of the use of the trained machine learning model for the prediction are identified in this description and in FIG. 4 with a subscript “P”, in order to distinguish them from the MRI images which are used or are generated in the case of the training of the machine learning model.

The patient data (PD) can comprise still further data, in particular if the training of the machine learning model has also taken place on the basis of further input data.

However, the patient data (PD) do not comprise any other/further MRI images besides the at least one first MRI image and optionally the at least one native MRI image.

The patient data (PD) are fed to the trained machine learning model (MLM^{t}). The machine learning model (MLM^{t}) is configured and trained to generate a second MRI image (MRI_{P}^{2*}) on the basis of the patient data (PD) and on the basis of the model parameters (MP) modified (learned) in the course of the training, wherein the second MRI image (MRI_{P}^{2*}) is a predicted MRI image of the liver or the part of the liver of the patient in the hepatobiliary phase after the administration of the hepatobiliary contrast agent.

The second MRI image (MRI_{P}^{2*}) can be displayed on a monitor, printed on a printer, stored on a data memory, and/or transmitted to a separate computer system.

The machine learning model can be for example an artificial neural network or comprise such a network.

An artificial neural network comprises at least three layers of processing elements: a first layer having input neurons (nodes), an Nth layer having at least one output neuron (node), and N−2 inner layers, where N is a natural number and is greater than 2.

The input neurons are used to receive the input data, in particular the at least one first MRI image and possibly the optional at least one native MRI image. For example, there can be one input neuron for each pixel or voxel of an MRI image if the representation is a real-space depiction in the form of a raster graphic. There may be additional input neurons for additional input values (e.g. information about the examination region, about the examination object, about conditions when the representation was generated, information about the state represented by the representation, and/or information about the point in time or the time span at/in which the representation was generated).

The output neurons can be used to output a predicted second MRI image, which represents the examination region at a point in time in the hepatobiliary phase.

The processing elements of the layers between the input neurons and the output neurons are connected to one another in a predetermined pattern with predetermined connection weights.

Preferably, the artificial neural network is a so-called convolutional neural network (CNN) or comprises such a network.

A CNN normally consists essentially of an alternately repeating array of filters (convolutional layer) and aggregation layers (pooling layer) terminating in one or more layers of fully connected neurons (dense/fully connected layer).

The training of the neural network may for example be carried out by means of a back propagation method. The aim is for the network to predict the second MRI image as reliably as possible. The quality of the prediction is described by a loss function. The goal is to minimize the loss function. In the case of the back propagation method, an artificial neural network is taught by altering the connection weights. In the trained state, the connection weights between the processing elements contain information with respect to the relationship between the at least one first MRI image and optionally the at least one native MRI image and the second MRI image, which can be used for prediction purposes.

A cross-validation method can be used to divide the data into training data sets and validation data sets. The training data set is used in the back propagation training of the network weights. The validation data set is used to check the accuracy of prediction with which the trained network can be applied to unknown data.

The artificial neural network can have an autoencoder architecture; e.g. the artificial neural network can have an architecture like the U-net (see e.g. O. Ronneberger et al.: U-net: Convolutional networks for biomedical image segmentation, International Conference on Medical image computing and computer-assisted intervention, pages 234-241, Springer, 2015, https://doi.org/10.1007/978-3-319-24574-4_28).

The artificial neural network can be a generative adversarial network (GAN) (see e.g. M.-Y. Liu et al.: Generative Adversarial Networks for Image and Video Synthesis: Algorithms and Applications, arXiv:2008.02793; J. Henry et al.: Pix2Pix GAN for Image-to-Image Translation, DOI: 10.13140/RG.2.2.32286.66887).

The artificial neural network can be a recurrent neural network or comprise such a network. Recurrent or feedback neural networks refer to neural networks that, in contrast to feed-forward networks, are distinguished by connections between neurons of one layer and neurons of the same layer or a preceding layer. The artificial neural network can comprise for example a long short-term memory (LSTM) (see e.g. Y. Gao et al.: Fully convolutional structured LSTM networks for joint 4D medical image segmentation, DOI: 10.1109/ISBI.2018.8363764).

The artificial neural network can be a transformer network (see e.g. D. Karimi et al.: Convolution-Free Medical Image Segmentation using Transformers, arXiv 2102.13645 [eess. IV]).

The invention can be performed wholly or partly with the aid of a computer system.

A “computer system” is an electronic data processing system that processes data by means of programmable calculation rules. Such a system typically comprises a “computer”, which is the unit that comprises a processor for carrying out logic operations, and peripherals.

In computer technology, “peripherals” denotes all devices that are connected to the computer and are used to control the computer and/or as input and output devices. Examples thereof are monitor (screen), printer, scanner, mouse, keyboard, drives, camera, microphone, speakers, etc. Internal ports and expansion cards are also regarded as peripherals in computer technology.

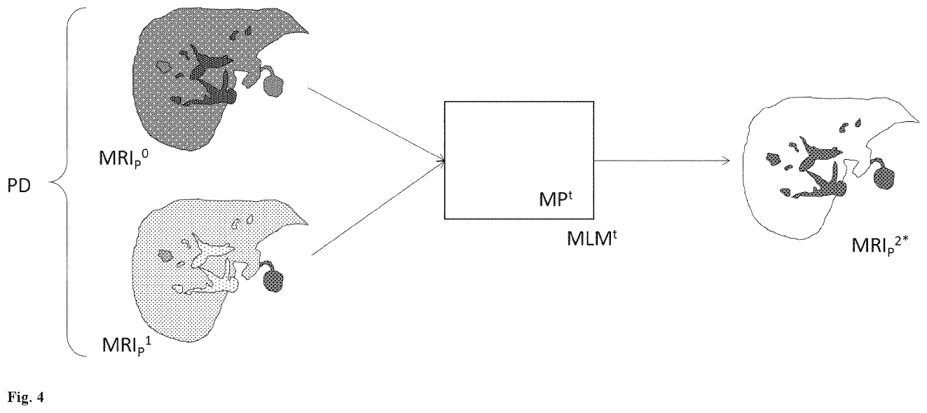

FIG. 5 shows by way of example and schematically an embodiment of the computer system according to the invention.

The computer system (10) shown in FIG. 5 comprises an input unit (11), a control and calculation unit (12) and an output unit (13).

The input unit (11) is used to receive data (e.g. patient data comprising one or more MRI images) and/or for the input of data and/or commands by a user of the computer system (10).

The output unit (13) is used to output data and/or information with respect to a user and/or to store data (e.g. the predicted MRI images) in a data memory and/or to transmit data to a separate computer system.

The control and calculation unit (12) is used for control of the computer system (10), for coordination of the data flows between the units of the computer system (10), and for the performance of calculations.

The control and calculation unit (12) is configured
to cause the input unit (11) to receive patient data, wherein the patient data comprise at least one MRI image, wherein the at least one MRI image is restricted to at least one first MRI image and optionally at least one native MRI image, wherein the at least one first MRI image represents a liver or a part of the liver of a patient at a point in time in the transitional phase after an administration of a hepatobiliary contrast agent, wherein the optional at least one native MRI image represents the liver or the part of the liver of the patient without contrast agent,
to input the patient data into a trained machine learning model, wherein the machine learning model was trained on the basis of training data to predict a second MRI image on the basis of at least one first MRI image and optionally at least one native MRI image, wherein the training data comprise input data and target data for each examination object of a plurality of examination objects,
wherein the input data comprise MRI images, wherein the MRI images are restricted to:
   at least one first MRI image, wherein the at least one first MRI image represents a liver or a part of the liver of the examination object at a point in time in the transitional phase after an administration of a hepatobiliary contrast agent, and
   optionally at least one native MRI image of the liver or the part of the liver of the examination object without contrast agent,
wherein the target data comprise a second MRI image, wherein the second MRI image represents the liver or the part of the liver of the examination object at a point in time in the hepatobiliary phase after the administration of the hepatobiliary contrast agent,
to receive a predicted MRI image from the trained machine learning model, wherein the predicted MRI image represents the liver or the part of the liver of the patient at a point in time in the hepatobiliary phase after the administration of the hepatobiliary contrast agent,
to cause the output unit (13) to output the predicted MRI image and/or to store it and/or to transmit it to a separate computer system.

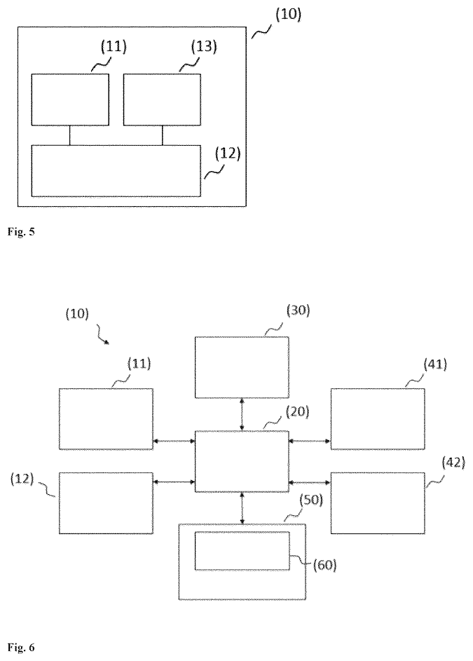

FIG. 6 shows by way of example and schematically a further embodiment of the computer system. The computer system (10) comprises a processing unit (20) connected to a memory (50).

The processing unit (20) may comprise one or more processors alone or in combination with one or more memories. The processing unit (20) can be customary computer hardware that is able to process information such as e.g. digital images, computer programs, and/or other digital information. The processing unit (20) normally consists of an arrangement of electronic circuits, some of which can be designed as an integrated circuit or as a plurality of integrated circuits connected to one another (an integrated circuit is sometimes also referred to as a “chip”). The processing unit (20) may be configured to execute computer programs that can be stored in a working memory of the processing unit (20) or in the memory (50) of the same or of a different computer system.

The memory (50) may be customary computer hardware that is able to store information such as digital images (for example representations of the examination region), data, computer programs and/or other digital information either temporarily and/or permanently. The memory (50) may comprise a volatile and/or nonvolatile memory and may be nonremovable or removable. Examples of suitable memories are RAM (random access memory), ROM (read-only memory), a hard disk, a flash memory, an exchangeable computer floppy disk, an optical disc, a magnetic tape, or a combination of the aforementioned. Optical discs can include compact discs with read-only memory (CD-ROM), compact discs with read/write function (CD-R/W), DVDs, Blu-ray discs, and the like.

The processing unit (20) may be connected not just to the memory (50), but also to one or more interfaces (11, 12, 30, 41, 42) in order to display, transmit and/or receive information. The interfaces may comprise one or more communication interfaces (11, 12) and/or one or more user interfaces (30, 41, 42). The one or more communication interfaces may be configured to send and/or receive information, for example to and/or from an MRI scanner, a CT scanner, an ultrasound camera, other computer systems, networks, data memories or the like. The one or more communication interfaces may be configured to transmit and/or receive information via physical (wired) and/or wireless communication connections. The one or more communication interfaces may comprise one or more interfaces for connection to a network, e.g. using technologies such as a cellphone, Wi-Fi, satellite, cable, DSL, optical fiber, and/or the like. In some examples, the one or more communication interfaces may comprise one or more close-range communication interfaces configured to connect devices with close-range communication technologies such as NFC, RFID, Bluetooth, Bluetooth LE, ZigBee, infrared (e.g. IrDA) or the like.

The user interfaces can comprise a display (30). A display (30) may be configured to display information to a user. Suitable examples thereof are a liquid crystal display (LCD), a light-emitting diode display (LED), a plasma display panel (PDP) or the like. The user input interface(s) (41, 42) may be wired or wireless and may be configured to receive information from a user in the computer system (10), for example for processing, storage and/or display. Suitable examples of user input interfaces are a microphone, an image or video recording device (for example a camera), a keyboard or a keypad, a joystick, a touch-sensitive surface (separate from a touchscreen or integrated therein) or the like. In some examples, the user interfaces may contain an automatic identification and data capture technology (AIDC) for machine-readable information. This can include barcodes, radiofrequency identification (RFID), magnetic strips, optical character recognition (OCR), integrated circuit cards (ICC) and the like. The user interfaces may furthermore include one or more interfaces for communication with peripheral devices such as printers and the like.

One or more computer programs (60) may be stored in the memory (50) and executed by the processing unit (20), which is thereby programmed to perform the functions described in this description. The retrieving, loading and execution of instructions of the computer program (60) may take place sequentially, such that an instruction is respectively retrieved, loaded and executed. However, the retrieving, loading and/or execution may also take place in parallel.

The machine learning model according to the invention may also be stored in the memory (50).

The computer system according to the invention may be designed as a laptop, notebook, netbook and/or tablet PC; it may also be a component of an MRI scanner, a CT scanner or an ultrasound diagnostic device.

The present invention also provides a computer program product. Such a computer program product includes a non-volatile data carrier, for example a CD, a DVD, a USB stick or another data storage medium. A computer program is stored on the data carrier. The computer program can be loaded into a working memory of a computer system (more particularly into a working memory of a computer system the present disclosure), where it causes the computer system to execute the following steps:
receiving patient data, wherein the patient data comprise at least one MRI image, wherein the at least one MRI image is restricted to at least one first MRI image and optionally at least one native MRI image, wherein the at least one first MRI image represents a liver or a part of the liver of a patient at a point in time in the transitional phase after an administration of a hepatobiliary contrast agent, wherein the optional at least one native MRI image represents the liver or the part of the liver of the patient without contrast agent,
inputting the patient data into a trained machine learning model, wherein the machine learning model was trained on the basis of training data to predict a second MRI image on the basis of at least one first MRI image and optionally at least one native MRI image, wherein the training data comprise input data and target data for each examination object of a plurality of examination objects,
wherein the input data comprise MRI images, wherein the MRI images are restricted to:
   at least one first MRI image, wherein the at least one first MRI image represents a liver or a part of the liver of the examination object at a point in time in the transitional phase after an administration of a hepatobiliary contrast agent, and
   optionally at least one native MRI image of the liver or the part of the liver of the examination object without contrast agent,
wherein the target data comprise a second MRI image, wherein the second MRI image represents the liver or the part of the liver of the examination object at a point in time in the hepatobiliary phase after the administration of the hepatobiliary contrast agent,
receiving a predicted MRI image from the trained machine learning model, wherein the predicted MRI image represents the liver or the part of the liver of the patient at a point in time in the hepatobiliary phase after the administration of the hepatobiliary contrast agent,
outputting and/or storing the predicted MRI image and/or transmitting the predicted MRI image to a separate computer system.

The computer program product can also be marketed in combination (in a set) with the contrast agent. Such a set is also referred to as a kit. Such a kit comprises the contrast agent and the computer program product. It is also possible for such a kit to comprise the contrast agent and means that allow the purchaser to obtain the computer program, for example by downloading it from a webpage. These means may include a link, i.e. an address of the webpage on which the computer program can be obtained, for example from which the computer program can be downloaded to a computer system connected to the internet. These means can comprise a code (e.g. an alphanumeric string or a QR code, or a DataMatrix code or a barcode or some other optically and/or electronically readable code) that gives the purchaser access to the computer program. Such a link and/or code may for example be printed on a packaging of the contrast agent and/or on a package leaflet of the contrast agent. A kit is thus a combination product comprising a contrast agent and a computer program (for example in the form of access to the computer program or in the form of executable program code on a data carrier) that are offered for sale together.

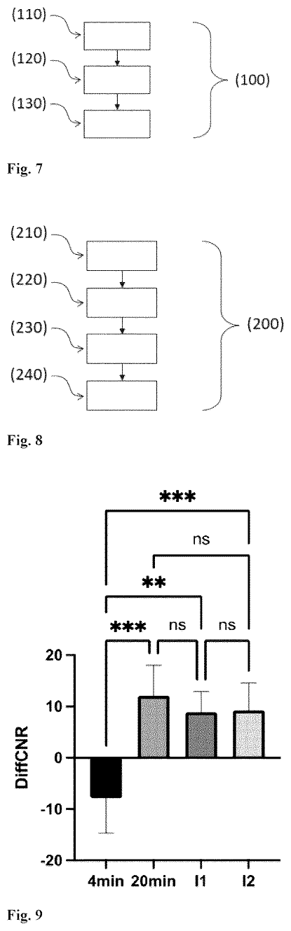

FIG. 7 shows one embodiment of the method for training the machine learning model in the form of a flowchart. The method (100) comprises the following steps:
(110) receiving and/or providing training data, wherein the training data comprise input data and target data for each examination object of a plurality of examination objects, wherein the input data comprise MRI images, wherein the MRI images are restricted to:
   at least one first MRI image, wherein the at least one first MRI image represents a liver or a part of the liver of the examination object at a point in time in the transitional phase after an administration of a hepatobiliary contrast agent, and
   optionally at least one native MRI image of the liver or the part of the liver of the examination object without contrast agent,
wherein the target data comprise a second MRI image, wherein the second MRI image represents the liver or the part of the liver of the examination object at a point in time in the hepatobiliary phase after the administration of the hepatobiliary contrast agent,
(120) training the machine learning model, wherein the machine learning model is configured to generate a predicted second MRI image on the basis of at least one first MRI image, optionally at least one native MRI image, and model parameters, wherein the training comprises, for each examination object of the plurality of examination objects:
   inputting the input data into the machine learning model,
   receiving a predicted second MRI image from the machine learning model,
   calculating a deviation between the second MRI image and the predicted second MRI image,
   modifying the model parameters with regard to reducing the deviation,
(130) storing and/or outputting the trained machine learning model and/or transmitting the trained machine learning model to a separate computer system and/or using the trained machine learning model for prediction.

FIG. 8 shows an embodiment of the computer-implemented method for using the trained machine learning model for predicting an MRI image in the form of a flowchart. The method (200) comprises the following steps:
(210) receiving patient data, wherein the patient data comprise at least one MRI image, wherein the at least one MRI image is restricted to at least one first MRI image and optionally at least one native MRI image, wherein the at least one first MRI image represents a liver or a part of the liver of a patient at a point in time in the transitional phase after an administration of a hepatobiliary contrast agent, wherein the optional at least one native MRI image represents the liver or the part of the liver of the patient without contrast agent,
(220) inputting the patient data into a trained machine learning model, wherein the machine learning model was trained on the basis of training data to predict a second MRI image on the basis of at least one first MRI image and optionally at least one native MRI image, wherein the training data comprise input data and target data for each examination object of a plurality of examination objects, wherein the input data comprise MRI images,
wherein the MRI images are restricted to:
   at least one first MRI image, wherein the at least one first MRI image represents a liver or a part of the liver of the examination object at a point in time in the transitional phase after an administration of a hepatobiliary contrast agent, and
   optionally at least one native MRI image of the liver or the part of the liver of the examination object without contrast agent,
wherein the target data comprise a second MRI image, wherein the second MRI image represents the liver or the part of the liver of the examination object at a point in time in the hepatobiliary phase after the administration of the hepatobiliary contrast agent,
(230) receiving a predicted MRI image from the trained machine learning model, wherein the predicted MRI image represents the liver or the part of the liver of the patient at a point in time in the hepatobiliary phase after the administration of the hepatobiliary contrast agent,
(240) outputting and/or storing the predicted MRI image and/or transmitting the predicted MRI image to a separate computer system.

### EXAMPLE

An exemplary implementation (11) for reducing the required waiting time for acquiring an MRI image of the liver of an examination object in the hepatobiliary phase is described hereinafter. The MRI image is predicted on the basis of at least one first MRI image, which represents the liver in the transitional phase, and at least one native MRI image.

The at least one first MRI image was recorded 4 minutes after administration of a hepatobiliary contrast agent (Primovist®).

The target MRI image (target, ground truth) was recorded 20 minutes after administration of the hepatobiliary contrast agent.

The exemplary implementation (11) is compared to a second implementation (12), in which, in addition to the at least one first MRI image and the at least one native MRI image, further MRI images from further phases were incorporated in the prediction of the MRI image of the liver of the examination object in the hepatobiliary phase.

The further MRI images were recorded 18 seconds (arterial phase), 65 seconds (portal-venous phase), and 95 seconds (late venous phase) after administration of the hepatobiliary contrast agent.

In order to obtain a training data set, 20 healthy Göttingen minipigs were subjected at three different occasions to an MRI using a standard dose (0.025 mmol/kg, gadoxetate disodium). Three of 19 animals were selected according to the random principal and retained for validation (18 examinations). One animal had to be excluded because of incomplete examinations. The remaining 16 animals (96 examinations) were used to train a GAN (CycleGAN) for an image-to-image conversion.

In the exemplary implementation (11), three layers were observed in the livers of the animals: X1, X2, X3. First MRI images and native images of the layers X1, X2, X3 were used as input data in order to predict the target Y.

The target Y to be predicted is located at the same position as the layer X2.

The target Y to be predicted is a water-only image of a T1-VIBE-DIXON sequence.

Three stacks of in each case five MRI images were generated for the layer X2, the layer X1 (above X2), and the layer X3 (below X2). Each of the image stacks contains the water-only image, the fat-only image, the in-phase and the opposed-phase of a T1-VIBE-DIXON sequence, which was recorded 4 minutes after injection of a liver-specific contrast agent (0.025 mmol/kg, gadoxetate) and a water-only image of a T1-VIBE-DIXON, which was recorded before the injection (a total of 15 MRI images).

As a comparative implementation (12), the water-only image of an arterial, portal-venous, and late-venous T1-VIBE-DIXON sequence of the layers X1, X2, X3 were additionally input. This results in a total number of 24 MRI images. All other settings correspond to the implementation I1. For the acquisition of the T1-VIBE-DIXON sequence, the following scanning parameters were used on a 1.5T Avanto Fit MRT Scanner (Siemens Healthcare GmbH): TR 6.9 ms, TE 2.39/4.77 ms, flip angle 10°, matrix 256x256, layer thickness 1.5 mm.

The machine learning models of the two implementations (CycleGAN in each case) were trained using 200 epochs and a linear learning rate drop, beginning after 100 epochs. The initial learning rate was defined at 0.0002, and a momentum of 0.5 was used. In addition, a batch size of 4 was used for each model. With regard to the network architecture, a ResNet 9 block generator having 64 filters in the last convolutional layer, transposition convolution as an upsampling method, and instance normalization was used. In addition, a PatchGAN was used as discriminator, which contains 64 filters in the first convolutional layer. During the training, an extensive data augmentation was used, including random trimming (224×224), random rotation (−15 to 15 degrees), and horizontal mirroring. To ensure the overall compatibility within each data stack, all MRI images were reduced to a size of 224×224 in order to correspond to the random trimming size.

To validate the efficiency of the network, ROI measurements were carried out in the abdominal aorta, the inferior vena cava, the portal vein, the liver parenchyma, and the autochthonus back musculature and the contrast to noise ratio (CNR) was calculated ((intensity target structure-intensity autochthonus back musculature)/standard deviation of the intensity of the back musculature).

Under the assumption that from point in time TP4 (see FIG. 2) to point in time TP5 to be predicted (see FIG. 2), the vascular CNR drops and the parenchymal CNR has to be raised, the difference of the hepatic CNR to the average of the vascular CNR was calculated as assessment metric (DiffCNR).

A significant rise was shown here of the DiffCNR of the transitional phase (4 minutes after contrast agent administration) to the real hepatobiliary contrast agent phase (average difference=−19.96; p=0.0005) and to the predicted MRI image of the implementation I1 (average difference=−16.76; p=0.0012) and 12 (average difference=−17.14; p=0.0003).

No significant difference was shown between the DiffCNR of the real hepatobiliary contrast agent phase and the predicted MRI images of the implementation I1 and I2. Moreover, no significant differences were shown between the predicted MRI images of the implementation I1 and I2.

The results of the DiffCNR are shown in the form of a graph of measurements in FIG. 9. In the graph, the arithmetic mean values and standard deviations of the DiffCNR values for the implementations I1 and I2 are shown. ns means “not significant”. ** stands for p<0.01. *** stands for p<0.001.

It was possible to show that to generate an MRI image of the liver or a part of the liver of an examination object, at least one MRI image of the liver or the part of the liver in the transitional phase and optionally at least one native MRI image of the liver or the part of the liver is sufficient.

Fewer resources are necessary due to the reduction in the number of MRI images. Fewer MRI images have to be generated to generate training data. The training of the machine learning model takes place faster and requires less data memory and computer power. This also applies analogously to the use of the trained machine learning model for the prediction. Since MRI images are generated only at at most two points in time for the training and the prediction, the generation of the MRI images is less unpleasant for the examination objects. They only have to stop movement at at most two points in time (in order to avoid movement artifacts). In this case, the two points in time are far enough away from one another (more than 3 minutes) that they can relax between these points in time.

1. A computer-implemented method, comprising:
providing a trained machine learning model (MLMt), wherein the trained machine learning model (MLMt) was trained on the basis of training data (TD) to generate a predicted second MRI image (MRI2*) on the basis of at least one first MRI image (MRI1) and optionally at least one native MRI image (MRI0), wherein the training data (TD) comprise input data (I) and target data (T) for each examination object of a plurality of examination objects;
wherein the input data (I) comprise MRI images (MRI0, MRI1), wherein the MRI images (MRI0, MRI1) are restricted to:
at least one first MRI image (MRI1), wherein the at least one first MRI image (MRI1) represents a liver or a part of the liver of the examination object at a point in time in the transitional phase after an administration of a hepatobiliary contrast agent; and
optionally at least one native MRI image (MRI0) of the liver or the part of the liver of the examination object without contrast agent;
wherein the target data (T) comprise a second MRI image (MRI2), wherein the second MRI image (MRI2) represents the liver or the part of the liver of the examination object at a point in time in the hepatobiliary phase after the administration of the hepatobiliary contrast agent;
receiving patient data (PD), wherein the patient data (PD) comprise at least one MRI image (MRIP0, MRIP1), wherein the at least one MRI image (MRIP0, MRIP1) is restricted to at least one first MRI image (MRIP1) and optionally at least one native MRI image (MRIP0), wherein the at least one first MRI image (MRIP1) represents a liver or a part of the liver of a patient at a point in time in the transitional phase after an administration of a hepatobiliary contrast agent, wherein the optional at least one native MRI image (MRIP0) represents the liver or the part of the liver of the patient without contrast agent;
inputting the patient data (PD) into the trained machine learning model (MLMt);
receiving a predicted MRI image (MRIP2*) from the trained machine learning model (MLMt), wherein the predicted MRI image (MRIP2*) represents the liver or the part of the liver of the patient at a point in time in the hepatobiliary phase after the administration of the hepatobiliary contrast agent; and
outputting and/or storing the predicted MRI image (MRIP2*) and/or transmitting the predicted MRI image (MRIP2*) to a separate computer system.

2. The method as claimed in claim 1, wherein the training of the machine learning model (MLMt) comprises:
receiving and/or providing the training data (TD);
training the machine learning model (MLM), wherein the machine learning model (MLM) is configured to generate a predicted second MRI image (MRI2*) on the basis of at least one first MRI image (MRI1), optionally at least one native MRI image (MRI0), and model parameters (MP), wherein the training comprises, for each examination object of the plurality of examination objects:
inputting the input data (I) into the machine learning model (MLM);
receiving a predicted second MRI image (MRI2*) from the machine learning model (MLM);
calculating a deviation between the second MRI image (MRI2) and the predicted second MRI image (MRI2*); and
modifying the model parameters (MP) with regard to reducing the deviation; and
storing and/or outputting the trained machine learning model (MLMt) and/or transmitting the trained machine learning model (MLMt) to a separate computer system and/or using the trained machine learning model (MLMt) for prediction.

3. The method as claimed in claim 1, wherein the examination object is a human.

4. The method as claimed in claim 1, wherein the at least one first MRI image (MRIP1, MRI1) represents the liver or the part of the liver of the patient/examination object 3 to 6 minutes after the administration of the contrast agent.

5. The method as claimed in claim 1, wherein the at least one first MRI image (MRIP1, MRI1) is at least one T1-weighted MRI image or comprises such an image.

6. The method as claimed in claim 1, wherein the at least one first MRI image (MRIP1, MRI1) is the result of a Dixon sequence.

7. The method as claimed in claim 1, wherein the at least one first MRI image (MRIP1, MRI1) comprises an in-phase image and/or an opposed-phase image and/or a fat-only image and/or a water-only image of the liver or the part of the liver.

8. The method as claimed in claim 1, wherein the input data (I) and/or patient data (PD) comprise at least one native MRI image (MRI0, MRIP0), wherein the at least one native MRI image (MRI0, MRIP0) is the result of T1-weighted Dixon sequence, phase

9. The method as claimed in claim 1, wherein the second MRI image (MRIP2, MRI2) is a representation of the liver at a point in time in the range of 15 minutes to 25 minutes, after the administration of the hepatobiliary contrast agent.

10. The method as claimed in claim 1, wherein the second MRI image (MRIP2, MRI2) is a T1-weighted MRI image.

11. The method as claimed in claim 1, wherein the contrast agent comprises:
the disodium salt of gadoxetic acid;
gadolinium 2,2′,2″-(10-{1-carboxy-2-[2-(4-ethoxyphenyl)ethoxy]ethyl}-1,4,7,10-tetraazacyclododecane-1,4,7-triyl)triacetate;
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate;
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate;
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate); or
gadolinium 2,2′,2″-{10-[(1S)-4-(4-butoxyphenyl)-1-carboxybutyl]-1,4,7,10-tetraazacyclododecane-1,4,7-triyl}triacetate.

12. A computer system, comprising:
an input unit;
a control and calculation unit; and
an output unit;
wherein the control and calculation unit is configured:
to cause the input unit to receive patient data (PD), wherein the patient data (PD) comprise at least one MRI image (MRIP0, MRIP1), wherein the at least one MRI image (MRIP0, MRIP1) is restricted to at least one first MRI image (MRIP1) and optionally at least one native MRI image (MRIP0), wherein the at least one first MRI image (MRIP1) represents a liver or a part of the liver of a patient at a point in time in the transitional phase after an administration of a hepatobiliary contrast agent, wherein the optional at least one native MRI image (MRIP0) represents the liver or the part of the liver of the patient without contrast agent;
to input the patient data (PD) into a trained machine learning model (MLMt), wherein the trained machine learning model (MLMt) was trained on the basis of training data (TD) to generate a predicted second MRI image (MRI2*) on the basis of at least one first MRI image (MRI1) and optionally at least one native MRI image (MRI0), wherein the training data (TD) comprise input data (I) and target data (T) for each examination object of a plurality of examination objects, wherein the input data (I) comprise MRI images (MRI0, MRI1);
wherein the MRI images (MRI0, MRI1) are restricted to:
at least one first MRI image (MRI1), wherein the at least one first MRI image (MRI1) represents a liver or a part of the liver of the examination object at a point in time in the transitional phase after an administration of a hepatobiliary contrast agent; and
optionally at least one native MRI image (MRI0) of the liver or the part of the liver of the examination object without contrast agent;
wherein the target data (T) comprise a second MRI image (MRI2), wherein the second MRI image (MRI2) represents the liver or the part of the liver of the examination object at a point in time in the hepatobiliary phase after the administration of the hepatobiliary contrast agent;
receiving a predicted MRI image (MRIP2*) from the trained machine learning model (MLMt), wherein the predicted MRI image (MRIP2*) represents the liver or the part of the liver of the patient at a point in time in the hepatobiliary phase after the administration of the hepatobiliary contrast agent; and
to cause the output unit (13) to output the predicted MRI image (MRIP2*) and/or to store it and/or to transmit it to a separate computer system.

13. A computer program product comprising a data carrier on which a computer program is stored that can be loaded into a working memory of a computer system, wherein it causes the computer system to execute the following:
receiving patient data (PD), wherein the patient data (PD) comprise at least one MRI image (MRIP0, MRIP1), wherein the at least one MRI image (MRIP0, MRIP1) is restricted to at least one first MRI image (MRIP1) and optionally at least one native MRI image (MRIP0), wherein the at least one first MRI image (MRIP1) represents a liver or a part of the liver of a patient at a point in time in the transitional phase after an administration of a hepatobiliary contrast agent, wherein the optional at least one native MRI image (MRIP0) represents the liver or the part of the liver of the patient without contrast agent;
inputting the patient data (PD) into a trained machine learning model (MLMt), wherein the machine learning model (MLMt) was trained on the basis of training data (TD) to generate a predicted second MRI image (MRI2*) on the basis of at least one first MRI image (MRI1) and optionally at least one native MRI image (MRI0), wherein the training data (TD) comprise input data (I) and target data (T) for each examination object of a plurality of examination objects, wherein the input data (I) comprise MRI images (MRI0, MRI1);
wherein the MRI images (MRI0, MRI1) are restricted to:
at least one first MRI image (MRI1), wherein the at least one first MRI image (MRI1) represents a liver or a part of the liver of the examination object at a point in time in the transitional phase after an administration of a hepatobiliary contrast agent; and
optionally at least one native MRI image (MRI0) of the liver or the part of the liver of the examination object without contrast agent;
wherein the target data (T) comprise a second MRI image (MRI2), wherein the second MRI image (MRI2) represents the liver or the part of the liver of the examination object at a point in time in the hepatobiliary phase after the administration of the hepatobiliary contrast agent;
receiving a predicted MRI image (MRIP2*) from the trained machine learning model (MLMt), wherein the predicted MRI image (MRIP2*) represents the liver or the part of the liver of the patient at a point in time in the hepatobiliary phase after the administration of the hepatobiliary contrast agent; and
outputting and/or storing the predicted MRI image (MRIP2*) and/or transmitting the predicted MRI image (MRIP2*) to a separate computer system.

14-16. (canceled)

17. The method as claimed in claim 2, wherein the examination object is a human, wherein the at least one first MRI image (MRIP1, MRI1) represents the liver or the part of the liver of the patient/examination object 3 to 6 minutes after the administration of the contrast agent, and wherein the second MRI image (MRIP2, MRI2) is a representation of the liver at a point in time in the range of 15 minutes to 25 minutes, after the administration of the hepatobiliary contrast agent.

18. The method as claimed in claim 2, wherein the examination object is a human, and wherein the at least one first MRI image (MRIP1, MRI1) comprises an in-phase image and/or an opposed-phase image and/or a fat-only image and/or a water-only image of the liver or the part of the liver.

19. The method as claimed in claim 2, wherein the examination object is a human, and wherein the at least one native MRI image (MRI0, MRIP0) is the result of a T1-weighted Dixon sequence using in-and opposed-phase recording technology.

20. The method as claimed in claim 2, wherein the examination object is a human, wherein the second MRI image (MRIP2, MRI2) is a T1-weighted MRI image.

21. The computer program product as claimed in claim 13, wherein the training of the machine learning model (MLMt) comprises:
receiving and/or providing the training data (TD);
training the machine learning model (MLM), wherein the machine learning model (MLM) is configured to generate a predicted second MRI image (MRI2*) on the basis of at least one first MRI image (MRI1), optionally at least one native MRI image (MRI0), and model parameters (MP), wherein the training comprises, for each examination object of the plurality of examination objects:
inputting the input data (I) into the machine learning model (MLM);
receiving a predicted second MRI image (MRI2*) from the machine learning model (MLM);
calculating a deviation between the second MRI image (MRI2) and the predicted second MRI image (MRI2*); and
modifying the model parameters (MP) with regard to reducing the deviation; and
storing and/or outputting the trained machine learning model (MLMt) and/or transmitting the trained machine learning model (MLMt) to a separate computer system and/or using the trained machine learning model (MLMt) for prediction.

22. The computer program product as claimed in claim 13, wherein the examination object is a human, wherein the at least one first MRI image (MRIP1, MRI1) represents the liver or the part of the liver of the patient/examination object 3 to 6 minutes after the administration of the contrast agent, and wherein the second MRI image (MRIP2, MRI2) is a representation of the liver at a point in time in the range of 15 minutes to 25 minutes, after the administration of the hepatobiliary contrast agent.

23. The computer program product as claimed in claim 13, wherein the examination object is a human, wherein the at least one native MRI image (MRI0, MRIP0) is the result of a T1-weighted Dixon sequence using in-and opposed-phase recording technology, and wherein the second MRI image (MRIP2, MRI2) is a T1-weighted MRI image.
